(19) United States
(12) Patent Application Publication
(10) Pub. No.: US 2016/0022558 A1
(43) Pub. Date: Jan. 28, 2016

(54) Hair Treatment Products and Methods for Straightening Hair

(71) Applicants: David Ben Kunin, Minneapolis, MN (US); Sherry Ann Herrity, New Prague, MN (US); Samuel John Brocato, New York, NY (US)

(72) Inventors: David Ben Kunin, Minneapolis, MN (US); Sherry Ann Herrity, New Prague, MN (US); Samuel John Brocato, New York, NY (US)

(21) Appl. No.: 14/341,247

(22) Filed: Jul. 25, 2014

(51) Int. Cl. A61K 8/64; A61K 8/34; A61K 8/44; A61K 8/49; A61Q 5/06; A61K 8/36

(52) U.S. Cl. CPC A61K 8/64; A61Q 5/06; A61K 8/36; A61K 8/447; A61K 8/4973; A61K 8/34

(57) ABSTRACT

Methods and hair treatment products for smoothing or straightening hair having a formulation including at least one reducing agent, at least one protein fraction, at least one emollient, at least one emulsifier; and an aqueous diluent. The hair treatment product having a viscosity of from about 1 to about 1000 cps and a pH of about 1.5 to about 3.

### FIELD OF THE INVENTION

The present invention relates to hair treatment products and methods for straightening or smoothing frizzy or curly hair. More specifically, the present invention relates to hair treatment products and methods of hair treatment using such products that do not include formaldehyde.

### BACKGROUND OF THE RELATED ART

Formaldehyde-based “Brazilian” or Keratin hair treatment products have gained popularity for straightening or smoothing frizzy, kinky or curly hair. Such hair treatment products, however, are known to often cause eye irritations, nose bleeds, respiratory problems and potentially debilitating diseases to stylists in hair salons who frequently come into contact with and/or inhale various chemical agents in such hair treatment products when they are applied or administered. Such hair treatment products are even banned in some countries due to their toxicity.

The present invention addresses problems and limitations associated with the related art.

### SUMMARY OF THE INVENTION

One preferred method of treating hair to smooth hair includes the steps of first procuring a flat iron and a hair treatment product as disclosed herein. Then, the hair is shampooed and rinsed. The hair is then preferably conditioned. The hair treatment product is then sprayed on the hair and then the hair is covered from about 20 to 45 minutes for processing. Due to the preferred formulation of the hair treatment product, including 8.55% glyoxcylic acid, the processing time is shortened.

Then, the hair will likely be matted and tangled due to processing so a detangling and conditioning solution is applied and then combed through the hair. The hair is dried and the flat iron is repeatedly passed over the sections of hair to be smoothened. Preferably, a flat iron having vibrating plates is used as it will more easily glide through the hair as compared to a flat iron with static plates. Finally, the hair is then rinsed to generally remove the hair treatment product residue from the hair. The detangling and conditioning solution is then again applied on and combed through the hair. The hair can then be dried and styled, as desired.

One preferred hair treatment product has a formulation including from about 1.0 to about 15.0 percent by weight of at least one reducing agent; from about 0.5 to about 20.0 percent by weight of at least one protein fraction at a 20 percent concentration; from about 1.0 to about 10.0 percent by weight of at least one emollient; from about 0.1 to about 5.0 percent by weight of at least one emulsifier; and greater than 1.0 percent by weight of an aqueous diluent; such that the hair treatment product has a viscosity of from about 1 to about 1000 cps. The preferred hair treatment product has a pH of about 1.5 to about 3.

These and other objectives and advantages of the invention will appear more fully from the following description, made in conjunction with the accompanying drawings. And, although the disclosure hereof may be detailed and exact, the embodiments herein disclosed are merely examples that may be embodied in other specific systems and, while preferred embodiments have been described, the details may be changed without departing from the inventions sought to be supported herein by the present disclosure.

### DETAILED DESCRIPTION OF THE PREFERRED EMBODIMENTS

Consumer demand for hair smoothing products and systems for the treatment of hair remains high. The present inventors have developed a hair treatment product and method that does not include formaldehyde and also is fast and easy for a salon stylist to apply. Although the following will be discussed in a salon context, having a stylist and client relationship, it will be understood that the invention is not intended to be limited to such scenarios. One preferred hair treatment product formulation is disclosed below in Table 1.

The preferred hair treatment product formulation includes from about 1.0 to about 15.0 percent, preferably from about 1 to about 10 percent by weight of a reducing agent. Preferred reducing agents are selected from the group including organic acids which may include glyoxylic acid, cysteine HCL, citric acid, glycolic acid, lactic acid, ascorbic acid and the like and combinations thereof. In preferred embodiments the reducing agent will be glyoxylic acid in an amount of from about 1 to about 15, preferably from about 4 to about 10, more preferably from about 6 to about 9, most preferably about 8.55 percent by weight.

The preferred hair treatment product formulation further includes from about 0.5 to about 20.0 percent, preferably from about 0.5 to about 15.0 percent by weight of protein fractions. Protein fractions are available undiluted, and combinations of wheat protein and sericin and preferred. Protein fractions are available undiluted and also in 20% and 50% concentrations. Protein fractions are believed to strengthen which act as cross-linking agents to bind to the hair to strengthen and protect the cuticle layer of the hair. Such protein fractions may include hydrolyzed wheat protein, silk protein, sericin, keratin protein and the like and combinations thereof.

The preferred hair treatment product formulation further includes from about 1.0 to about 10.0 percent, preferably from about 1.0 to about 5.0 percent by weight of emollients, which soften, moisturize and lubricate the hair. Preferred emollients include polyols such as glycerin, sorbitol, propylene glycol, caprylyl glycol, propanediol and the like and combinations thereof.

The preferred hair treatment product formulation further includes from about 0.1 to about 5.0 percent, preferably from about 0.1 to about 3.0 percent by weight of emulsifiers. Preferred emulsifiers include PEG-40 Hydrogenated Castor Oil, PPG-5 Ceteth 20, PEG-60 Almond Glycerides, PPG-2 Hydroxyethyl Cocamide, Polysorbate 20, Polysorbate 80, Ceteareth-20 and the like and combinations thereof.

The preferred hair treatment product formulation further includes an aqueous diluent in a percentage by weight greater than from about 1.0 percent, preferably greater than about 30.0 percent, most preferably greater than from about 50 percent by weight. The preferred diluent is water. The dilutent is added such that the product is in liquid form and can be sprayed. This provides for enhanced ease of application, which saves the stylist significant time in applying the hair treatment.

Preferably, the hair treatment product has a viscosity that allows the hair treatment product to be sprayed from a spray bottle; preferably a viscosity of from about 1 to about 1000 cps, more preferably, from about 1 to about 200 cps. The preferred formulation, as disclosed in Example 1, has a viscosity of about 1 cps such that it can easily be sprayed onto the hair. In preferred embodiments the spray formulation of the hair treatment product will be packaged in a pump type spray bottle, but it can also be packaged in other type of spray bottles such as trigger spray style bottles, aerosol spray bottle and the like. One preferred bottle is 100ML Sharp Shoulder 24-410 HDPE cyclinder 3oz bottle model number 1C100-W14SS by Unique Blow Molding 341 Edgeley, Concord, ON L4K 3Y2, Canada.

The preferred hair treatment product formulation further includes fragrances that can be used to mask odor of various product ingredients and the result of the hair treatment product reaction with the hair. The percentage by weight of such odor masking fragrances is from about 0.1 to about 10.0, preferably from about 0.1 to about 4.0 percent by weight. When used in this concentration, the hair treatment product has substantially no odor, as compared to known smoothing hair treatment products that are believed to have a significant displeasing aroma. One preferred masking fragrance is Ordenone® manufactured by Belle Aire Fragrances of Mundelein, Ill. It will be understood that this is one example of a suitable masking fragrance and that the invention is not intended to be limited to any one masking fragrance.

The preferred hair treatment product formulation further includes from about 0.1 to about 5.0, preferably from about 0.1 to about 2.0 percent by weight of conditioning agents. Preferred conditioning agents include Polyquaternium 4, Polyquaternium-7, Polyquaternium-10, Polyquaternium 37, Polyquaternium 39, Cetrimonium Chloride, Behentrimonium Chloride, Stearalkonium Chloride, Guar Hydroxypropyltrimonium Chloride or the like and combinations thereof.

The preferred pH of the hair treatment product is from about 1.5 to about 3, more preferably, from about 1.65 to about 2.5; more preferably from about 1.8 to about 2.1; most preferably about 1.9. The pH of the formulation may be adjusted with agents including, but not limited to, Sodium Hydroxide, Triethanolamine, Aminomethyl Propanol and the like and combinations thereof. This may require a hair treatment product formulation having a pH adjusting agent percentage by weight of from about 0.1 to about 5.0, preferably from about 0.1 to about 3.0 percent.

TABLE 1
ONE SET OF PREFERRED HAIR TREATMENT
PRODUCT FORMULATION RANGES

| | Percent by | Most Preferred |
|---|---|---|
| Ingredient | Weight | Percent by Weight |
| Reducing agents | 1.0-15.0%, | 1.0-10.0% |
| Protein fractions | 0.5-20.0%, | 0.5-15.0% |
| Emollients | 1.0-10.0%, | 1.0-5.0% |
| Emulsifiers | 0.1-5.0%, | 0.1-3.0% |
| Aqueous Diluent | Greater than | preferably greater |
| | 1.0% | than 30.0%, most |
| | | preferably greater |
| | | than 50.0% |
| Fragrances | 0.1-10.0% | 0.1-4.0% |
| Conditioning agents | 0.1-5.0% | 0.1-2.0% |
| pH adjusting agents | 0.1-5.0% | 0.1-3.0% |

One preferred hair treatment product formulation is disclosed below in Example 1.

### EXAMPLE 1

### Hair Treatment Product Formulation

| | | Formula | Percent | Adjusted |
|---|---|---|---|---|
| | INCI Name | Percent | Activity | Quantity |
| Trade Name | | | | |
| Water | Water | 58.00 | 100.000 | 58.00000 |
| Glycerin | Glycerin | 5.00 | 100.000 | 5.00000 |
| SERISEAL | Water | 1.50 | 72.875 | 1.09313 |
| | Polyquaternium-7 | 1.50 | 20.000 | 0.30000 |
| | Sericin | 1.50 | 2.750 | 0.04125 |
| | Guar Hydroxypropyl- | 1.50 | 0.775 | 0.01163 |
| | trimonium Chloride | | | |
| | Cetrimonium Chloride | 1.50 | 1.000 | 0.01500 |
| | Behentrimonium | 1.50 | 1.000 | 0.01500 |
| | Chloride | | | |
| SERILISS | Water | 30.00 | 38.200 | 11.46000 |
| | Polycarbamide | 30.00 | 32.500 | 9.75000 |
| | Proteins | | | |
| | Glyoxylic Acid | 30.00 | 28.500 | 8.55000 |
| | Sericin | 30.00 | 0.500 | 0.15000 |
| | Guar Hydroxypropyl- | 30.00 | 0.300 | 0.09000 |
| | trimonium Chloride | | | |
| Triethanolamine | Triethanolamine | 1.50 | 100.000 | 1.50000 |
| Cremophor | PEG-40 Hydrogenated | 2.00 | 100.000 | 2.00000 |
| RH-40 | Castor Oil | | | |
| Ordenone ® | Fragrance | 2.00 | 100.000 | 2.00000 |
| Physical | | | | |
| Properties | | | | |
| pH | About 1.9 | | | |
| Viscosity | About 1 cps | | | |

Preferred methods of straightening hair include the following steps. First the hair is washed or shampooed at least once, preferably twice in order to clean the hair in preparation for treatment. The preferred shampoo is SUPERSILK Detoxify from Beautopia LLC of Minneapolis, Minn., but it will be appreciated that other shampoos can be used. Next, the hair is preferably conditioned with Beautopia LLC SUPERSILK Rinse-out Conditioner from Beautopia LLC of Minneapolis, MN and then rinsed with water. It will be appreciated, however, that other conditioners can be used in alternate embodiments of the present invention. The hair is then towel dried and parted into about four sections. The hair treatment product described in Example 1 is placed in a pump type spray bottle for application to the hair. Then, starting at the nape of the neck, the hair treatment product is sprayed onto approximately ¼ inch sections of the hair from the scalp to the ends of the hair so that the entire length of each hair is bathed in the treatment product. The hair treatment product is then worked through the recently sprayed hair with a comb and the stylists' fingers. This process of spraying the hair treatment product on each hair, from the root of the hair to the end of the hair in groups of hair strands, is preferably repeated for all sections of the hair that are desired to be straightened. Then, the hair is preferably covered with an air restricting plastic bag or the like to retain moisture and maintain temperature. The hair treatment product is preferably left on the hair to process for from about 20 minutes for fine, fragile hair, preferably to about 30 minutes for medium textured, wavy to frizzy hair and preferably to about 45 minutes for resistant, curly, kinky or very frizzy hair. The inventors found that hair treatment products preferably having at least 8.55% by weight of a reducing agent, preferably glyocylic acid, is believed to treat the hair much faster than those having lower concentrations and that this is one significant improvement over known methods and products. After the preferred processing time has elapsed, the air restricting plastic bag is removed and a preferred conditioning and detangling leave-in cream, described in Table 2, below, is applied to the hair and preferably worked into the hair. Preferably, at this stage, the hair will be matted and substantially detangled with a comb, a brush and/or the stylist's fingers. In preferred embodiments, additional conditioning and detangling cream is applied to the hair until the stylist is able to complete the detangling of the hair and a comb can be passed through all of the hair that is being treated. The preferred conditioning and detangling cream is SUPERSILK Pure Indulgence Leave-In Treatment from Beautopia LCC of Minneapolis, Minn., which also includes about 0.0300% silk amino acid proteins and 0.00688% sericin. The conditioning and detangling leave-in solution is preferably worked into the hair and combed through the hair for even distribution and to prepare the hair for blow drying. Then the hair is dried, preferably with a blow dryer. After the hair is dry, a flat iron , preferably a flat iron pre-heated to from about 370 degrees F. for fine, fragile or medium wavy to frizzy hair, to about 410 degrees F. for resistant, curly, kinky or very frizzy hair, is used to straighten the hair. In preferred embodiments, the flat iron will be a vibrating flat iron. The flat iron is preferably used to straighten the hair by starting at the nape of the neck, where a selected group of hair strands, preferably the hair strands extending away from the skin in an area of from about 1/16th of an inch to about 3/16th of an inch, preferably about an ⅛^{th} of an inch are engaged between the respective plates of the flat iron and the hair is then passed between the hot plates so that the temperature of each the respective hair strands is raised over the entire length of each hair strand. In preferred embodiments, each 118^{th} inch section of hair strands will be engaged with a vibrating flat iron pre heated to from about 370 to about 410 degrees F., and passed through the flat iron from 5 times for fine, fragile hair, to 7 times for medium texture, wavy to frizzy hair, to as many as 7 to 10 times for resistant, curly, kinky or very frizzy hair. The preferred flat iron is a flat iron having vibrating plates, such as the VIBRASTRAIT® flat iron from Beautopia LLC of Minneapolis, Minn. It is believed by the inventors that vibrating flat iron substantially increases the ease with which the plates of the flat iron glide over the hair and further aids in subsequent detangling and straightening the hair. It is believed that use of such vibrating flat irons or straightening irons are a significant improvement in such hair straightening processes. Following the hair straightening step in which the hair strands are passed through the flat iron, the hair may have a chalky product residue that is present on the hair. The hair is then rinsed thoroughly to remove any such product residue. In preferred embodiments, the hair is then treated again with the preferred conditioning and detangling leave-in cream described above. After applying the cream, it is combed into and through the hair. The hair is then blow dried again and styled, as desired.

TABLE 2
PREFERRED CONDITIONING AND DETANGLING
CREAM FORMULATION

| | | | | Adjusted |
|---|---|---|---|---|
| | | Formula | Percent | Quantity |
| Trade Name | INCI Name | Percent | Activity | Percent |
| Water | Water | 91.05 | 100.000 | 91.05000 |
| Natrosol 250 | Hydroxyethylcellulose | 0.20 | 100.000 | 0.20000 |
| HHR CS | | | | |
| Dissolvine NA2 | Disodium EDTA | 0.05 | 100.000 | 0.05000 |
| Glucam E-20 | Methyl Gluceth-20 | 0.50 | 100.000 | 0.50000 |
| Pure Thix 1442 | Polyether-1 | 1.25 | 100.000 | 1.25000 |
| RITA Cetearyl | Cetearyl Alcohol | 3.20 | 100.000 | 3.20000 |
| Alcohol 50/50 | | | | |
| Varisoft BT-85 | Behentrimonium | 0.85 | 100.000 | 0.85000 |
| | Chloride | | | |
| Macadamia | Macadamia Ternifolia | 0.50 | 100.000 | 0.50000 |
| Seed Oil | Seed Oil | | | |
| Ritasil 1501 | Cyclopentasiloxane | 0.75 | 85.000 | 0.63750 |
| | Dimethiconol | 0.75 | 15.000 | 0.11250 |
| CE-8411 Smooth | Bis-Isopropylamino- | 0.50 | 56.000 | 0.28000 |
| Plus Emulsion | PG-Propyl Dimethi- | | | |
| | cone/Bis-Isobutyl | | | |
| | PEG-14 Copolymer | | | |
| | Polysorbate 20 | 0.50 | 37.000 | 0.18500 |
| | Butyloctanol | 0.50 | 7.000 | 0.03500 |
| SERISEAL | Water | 0.25 | 72.875 | 0.18219 |
| | Polyquaternium-7 | 0.25 | 20.000 | 0.05000 |
| | Sericin | 0.25 | 2.750 | 0.00688 |
| | Guar Hydroxypro- | 0.25 | 0.775 | 0.00194 |
| | pyltrimonium | | | |
| | Chloride | | | |
| | Cetrimonium Chloride | 0.25 | 1.000 | 0.00250 |
| | Behentrimonium | 0.25 | 1.000 | 0.00250 |
| | Chloride | | | |
| Crosilkquat- | Water | 0.15 | 72.875 | 0.10931 |
| LQ-WD | | | | |
| | Cocodimonium | 0.15 | 20.000 | 0.03000 |
| | Hydroxypropyl Silk | | | |
| | Amino Acids | | | |
| Euxyl K-713 | Potassium Sorbate | 0.50 | 50.000 | 0.25000 |
| | Sodium Benzoate | 0.50 | 50.000 | 0.25000 |
| F-(Lebermuth) | Fragrance | 0.25 | 67.340 | 0.16835 |
| Supersilk | | | | |
| 93-0119-92 | | | | |

### EXAMPLE 2

### Hair Treatment Method: Fine, Fragile Hair

One preferred method of straightening fine, fragile hair includes the following steps.

The hair is washed with shampoo and the shampoo is then rinsed out with water. Next, a conditioning agent is applied to the hair and the hair is then rinsed to remove the conditioning agent and any remaining shampoo. The hair is then towel dried and parted into about four sections. Then, starting at the nape of the neck and using approximately ¼ inch sections of hair, the hair treatment product described above in Example 1 above is sprayed onto the hair from the roots of the hair at the scalp to the ends of the hair. The hair treatment product is then worked through the recently sprayed hair with a comb and the stylist's fingers. This process is repeated for all sections of the hair that are desired to be straightened. Then, the hair is covered with an air restricting plastic bag or the like to retain moisture. The hair treatment product is left on the hair for approximately 20 minutes to process. After the processing time has elapsed, the plastic bag is removed and the preferred conditioning and detangling leave-in cream is apply to the hair. Typically, at this stage, the hair will be matted and substantially tangled. Additional the conditioning and detangling leave-in cream can be applied to the hair and worked into the hair until the stylist is able to detangle the hair. The conditioning and detangling leave-in cream is combed through the hair for even distribution and to prepare the hair for blow drying. The hair is then dried with a blow dryer. A flat iron having vibrating plates is then pre-heated to about 370 degrees F. Starting at the nape of the neck, the flat iron is passed over ⅛ inch sections of hair with the hot flat iron. This straightening step is repeated 4 times and then these straightening steps are repeated for each ⅛ inch section of hair. Then, the hair is rinsed thoroughly to remove any product residue. Next, the preferred conditioning and detangling leave-in cream is reapplied and combed through the hair and the hair is then blow dried again and styled, as desired.

### EXAMPLE 3

### Hair Treatment Method: Medium Textured, Wavy to Frizzy Hair

One method of straightening medium textured, wavy to frizzy hair includes the following steps. The hair is washed with shampoo and the shampoo is then rinsed out with water. Next, a conditioning agent is applied to the hair and the hair is then rinsed to remove the conditioning agent and any remaining shampoo. The hair is then towel dried and parted into about four sections. Then, starting at the nape of the neck and using approximately ¼ inch sections of hair, the hair treatment product described above in Example 1 above is sprayed onto the hair from the roots of the hair at the scalp to the ends of the hair. The hair treatment product is then worked through the recently sprayed hair with a comb and the stylist's fingers. This process is repeated for all sections of the hair that are desired to be straightened. Then, the hair is covered with an air restricting plastic bag or the like to retain moisture. The hair treatment product is left on the hair for approximately 30 minutes to process. After the processing time has elapsed, the plastic bag is removed and the preferred conditioning and detangling leave-in cream is apply to the hair. Typically, at this stage, the hair will be matted and substantially tangled. Additional the conditioning and detangling leave-in cream can be applied to the hair and worked into the hair until the stylist is able to detangle the hair. The conditioning and detangling leave-in cream is combed through the hair for even distribution and to prepare the hair for blow drying. The hair is then dried with a blow dryer. A flat iron having vibrating plates is then pre-heated to about 370 degrees F. Starting at the nape of the neck, the flat iron is passed over ⅛ inch sections of hair with the hot flat iron. This straightening step is repeated 6 times and then these straightening steps are repeated for each ⅛ inch section of hair. Then, the hair is rinsed thoroughly to remove any product residue. Next, the preferred conditioning and detangling leave-in cream is reapplied and combed through the hair and the hair is then blow dried again and styled, as desired.

### EXAMPLE 4

### Hair Treatment Method: Resistant, Curly, Kinky or Very Frizzy Hair

One method of straightening resistant, curly, kinky or very frizzy hair includes the following steps. The hair is washed with shampoo and the shampoo is then rinsed out with water. Next, a conditioning agent is applied to the hair and the hair is then rinsed to remove the conditioning agent and any remaining shampoo. The hair is then towel dried and parted into about four sections. Then, starting at the nape of the neck and using approximately ¼ inch sections of hair, the hair treatment product described above in Example 1 above is sprayed onto the hair from the roots of the hair at the scalp to the ends of the hair. The hair treatment product is then worked through the recently sprayed hair with a comb and the stylist's fingers. This process is repeated for all sections of the hair that are desired to be straightened. Then, the hair is covered with an air restricting plastic bag or the like to retain moisture. The hair treatment product is left on the hair for approximately 45 minutes to process. After the processing time has elapsed, the plastic bag is removed and the preferred conditioning and detangling leave-in cream is apply to the hair. Typically, at this stage, the hair will be matted and substantially tangled. Additional the conditioning and detangling leave-in cream can be applied to the hair and worked into the hair until the stylist is able to detangle the hair. The conditioning and detangling leave-in cream is combed through the hair for even distribution and to prepare the hair for blow drying. The hair is then dried with a blow dryer. A flat iron having vibrating plates is then pre-heated to about 410 degrees F. Starting at the nape of the neck, the flat iron is passed over ⅛ inch sections of hair with the hot flat iron. This straightening step is repeated from 6 to 10 times and then these straightening steps are repeated for each ⅛ inch section of hair. Then, the hair is rinsed thoroughly to remove any product residue. Next, the preferred conditioning and detangling leave-in cream is reapplied and combed through the hair and the hair is then blow dried again and styled, as desired.

When the preferred hair treatment products are used in accordance with the methods disclosed herein, frizz can be reduced by up to about 90 to about 100 percent, curls can be smoothed up to about 90 percent and hair drying time can be reduced up to about 60 percent for about 8-12 weeks. It is further believed that the preferred hair treatment products and methods will not substantially remove processed color from hair. As will be appreciated, the majority of straightening treatments on the market will remove color from color treated hair due to at least in part to their glyoxcylic content.

A further method of treating hair includes the following steps. First the hair is preferably washed with shampoo at least once, preferably twice. The preferred shampoo is SUPERSILK Detoxify from Beautopia LCC of Minneapolis, Minn. Following the washing, the hair is rinsed out with water. Next, the hair is conditioned with Beautopia SUPERSILK Rinse-out Conditioner from Beautopia LLC of Minneapolis, Minn. The hair is rinsed and then towel tried and parted into about four sections. Next, the hair treatment product described above in Example 1 above is applied to each hair section by spraying. The inventors found that it is most important to apply the hair treatment product to the mid-lengths and ends. The hair treatment product is then combed through the hair, preferably with the wide side of a traditional comb. Then, the detangling and conditioning cream described above is applied to the hair, preferably SUPERSILK Pure Indulgence Leave-In Treatment from Beautopia LCC of Minneapolis, Minn. The detangling and conditioning cream is worked into the hair and allowed to act on the hair for from about 15 to about 60 minutes, preferably from about 20-45 minutes, perhaps while the stylist is cutting the client's hair. After the time in which the detangling and conditioning cream is allowed to act on the hair has elapsed, a hot flat iron is then used to straighten the hair in small sections using from about 1 to about 3 passes of the flat iron, most preferably with a vibrating flat iron, with a temperature no higher than about 380 degrees F. This method produces smoother hair that will last about 10 days. As will be appreciated, after the straightening step, there will likely be a chalky product residue on the hair. This residue can be removed by rinsing the hair, then the detangling and conditioning cream is applied to the hair again that the hair is dried and styled, as desired.

It is to be understood, however, that even though numerous characteristics and advantages of the present invention have been set forth in the foregoing description, together with details of the specific steps and function of the invention, the disclosure is illustrative only, and changes may be made in detail, especially in matters of amount, degree, length of time and arrangement of appropriate steps within the principles of the invention to the full extent indicated by the broad general meaning of the terms in which the present invention is expressed.

What is claimed is:

1. A hair treatment product including:
from about 1.0 to about 15.0 percent by weight of at least one reducing agent;
from about 0.5 to about 20.0 percent by weight of at least one protein fraction;
from about 1.0 to about 10.0 percent by weight of at least one emollient;
from about 0.1 to about 5.0 percent by weight of at least one emulsifier;
from about 0.1 to about 10.0 percent by weight of a masking fragrance;
greater than 1.0 percent by weight of an aqueous diluent; such that the hair treatment product has a viscosity of about 1 to about 1000 cps;
wherein the hair treatment product has a pH of about 1.5 to about 3.

2. The hair treatment product of claim 1, wherein the reducing agents are selected from the group consisting of glyoxylic acid, cysteine HCL, citric acid, glycolic acid, lactic acid, ascorbic acid and combinations thereof.

3. The hair treatment product of claim 1, wherein the protein fractions are selected from the group consisting of hydrolyzed wheat protein, silk protein, sericin, keratin protein and combinations thereof.

4. The hair treatment product of claim 1, wherein the emollients are selected from the group consisting of glycerin, sorbitol, propylene glycol, caprylyl glycol, propanediol and combinations thereof.

5. The hair treatment product of claim 1, wherein the emulsifiers are selected from the group consisting of PEG-40 Hydrogenated Castor Oil, PPG-5 Ceteth 20, PEG-60 Almond Glycerides, PPG-2 Hydroxyethyl Cocamide, Polysorbate 20, Polysorbate 80, Ceteareth-20 and combinations thereof.

6. The hair treatment product of claim 1, wherein the viscosity of the hair treatment product is less than about 200 cps.

7. The hair treatment product of claim 1, wherein the viscosity of the hair treatment product is about 1.9 cps.

8. The hair treatment product of claim 1, including a reducing agent is 8.55% glyoxcylic acid.

9. A hair treatment product including:
from about 1.0 to about 10.0 percent by weight of at least one reducing agent;
from about 0.5 to about 15.0% percent by weight of at least one protein fraction;
from about 1.0 to about 10.0 percent by weight of at least one emollient;
from about 0.1 to about 3.0 percent by weight of at least one emulsifier;
from about 0.1 to about 4.0 percent by weight of a masking fragrance;
greater than 1.0 percent by weight of an aqueous diluent; such that the hair treatment product has a viscosity of about 1 to about 1000 cps;
wherein the hair treatment product has a pH of about 1.5 to about 3.

10. The hair treatment product of claim 9, wherein the reducing agents are selected from the group consisting of glyoxylic acid, cysteine HCL, citric acid, glycolic acid, lactic acid, ascorbic acid and combinations thereof.

11. The hair treatment product of claim 9, wherein the protein fractions are selected from the group consisting of hydrolyzed wheat protein, silk protein, sericin, keratin protein and combinations thereof.

12. The hair treatment product of claim 9, wherein the emollients are selected from the group consisting of glycerin, sorbitol, propylene glycol, caprylyl glycol, propanediol and combinations thereof.

13. The hair treatment product of claim 9, wherein the emulsifiers are selected from the group consisting of PEG-40 Hydrogenated Castor Oil, PPG-5 Ceteth 20, PEG-60 Almond Glycerides, PPG-2 Hydroxyethyl Cocamide, Polysorbate 20, Polysorbate 80, Ceteareth-20 and combinations thereof.

14. The hair treatment product of claim 9, wherein the viscosity of the hair treatment product is less than about 200 cps.

15. The hair treatment product of claim 14, wherein the viscosity of the hair treatment product is about 1.9 cps.

16. A method of treating hair comprising the steps of:
providing a hair treatment product, the hair treatment product including from about 1.0 to about 15.0 percent by weight of at least one reducing agent;
from about 0.5 to about 20.0 percent by weight of at least one protein fraction;
from about 1.0 to about 10.0 percent by weight of at least one emollient; from about 0.1 to about 5.0 percent by weight of at least one emulsifier; from about 0.1 to about 4.0 percent by weight of a masking fragrance; and greater than 1.0 percent by weight of an aqueous diluent; such that the hair treatment product has a viscosity of from about 1 to about 1000 cps; wherein the hair treatment product has a pH of about 1.5 to about 3;
washing the hair with shampoo;
rinsing the hair with water;
spraying the hair with the hair treatment product;
covering the hair with an air restricting covering for a period of time of from about 20 to about 45 minutes;
uncovering the hair;
applying a detangling and conditioning formulation to the hair;
drying the hair;
passing a hot flat iron over separate sections of the hair and repeating this step until the hot flat iron has been passed over each section of the hair more than once; wherein the flat iron is heated to a temperature of from about 370 to about 410 degrees F.;
rinsing the hair to remove any residue from the hair treatment product;
applying the detangling and conditioning formulation to the hair; and
drying the hair.

17. The method of claim 16, wherein the flat iron is passed over each section of the hair from about 5 to about 10 times.

18. The method of claim 17, wherein the flat iron is heated to a temperature of between about 370 to about 410 degrees F.

19. The method of claim 18, wherein the flat iron is a vibrating flat iron.

20. The method of claim 16, wherein the reducing agent of the hair treatment product is glyoxcylic acid.
